(19) United States
(12) Patent Application Publication
(10) Pub. No.: US 2007/0043099 A1
(43) Pub. Date: Feb. 22, 2007

(54) Crystalline forms of carvedilol and processes for their preparation

(76) Inventors: Igor Lifshitz, Petach-Tiqva (IL); Shlomit Wizel, Petach-Tiqva (IL)

Correspondence Address: KENYON & KENYON LLP, ONE BROADWAY, NEW YORK, NY, 10004, US

(21) Appl. No.: 11/450,699

(22) Filed: Jun. 9, 2006

Related U.S. Application Data
(60) Provisional application No. 60/689,776, filed on Jun. 9, 2005.

(51) Int. Cl. A61K 31/403; C07D 209/82

(52) U.S. Cl. 514/411; 548/444

(57) ABSTRACT

This invention relates to a novel crystalline form of carvedilol, to processes for its preparation, to compositions containing it and to its use in medicine. This invention further relates to a novel process for preparing crystalline carvedilol Form II.

### RELATED APPLICATIONS

This application claims the benefit of U.S. provisional application No. 60/689,776, filed Jun. 9, 2005; herein incorporated by reference.

### FIELD OF THE INVENTION

This invention relates to crystalline forms of carvedilol.

### BACKGROUND OF THE INVENTION

Carvedilol, (±)-1-(Carbazol-4-yloxy)-3-[[2-(o-methoxyphenoxy)ethyl]amino]-2-propanol, is a nonselective β-adrenergic blocker with α₁-blocking activity. Carvedilol is a racemic mixture having the following structural formula:

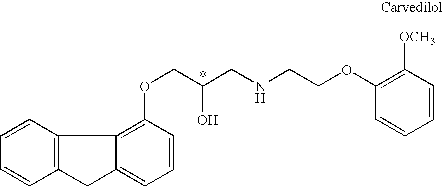

Carvedilol is the active ingredient of COREG®, which is indicated for the treatment of congestive heart failure and for the management of hypertension. Since carvedilol is a multiple-action drug, its beta-blocking activity affects the response to certain nerve impulses in parts of the body. As a result, beta-blockers decrease the heart's need for blood and oxygen by reducing its workload. Carvedilol is also known to be a vasodilator resulting primarily from alpha-adrenoceptor blockade. The multiple actions of carvedilol are responsible for the antihypertensive efficacy of the drug and for its effectiveness in managing congestive heart failure.

International application No. WO 99/05105 discloses carvedilol polymorphic forms designated Form I and Form II.

International application No. WO 02/00216 discloses carvedilol polymorphic forms designated Form III and Form IV. Also disclosed is Form V or a solvate thereof, with methyl-ethyl ketone.

International application No. WO 03/059807 discloses carvedilol polymorphic form designated Form VI or a solvate thereof, and process for its preparation with ethyl acetate including a seeding step.

The present invention relates to the solid state physical properties of carvedilol.

These properties can be influenced by controlling the conditions under which carvedilol is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry or infrared spectrometry.

The present invention also relates to solvates of carvedilol. When a substance crystallizes out of solution, it may trap molecules of solvent at regular intervals in the crystal lattice. Solvation also affects utilitarian physical properties of the solid state like flowability and dissolution rate.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties determine whether crystals of the form will adhere to each other when compacted into a tablet.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

### SUMMARY OF THE INVENTION

The present invention provides a crystalline form of carvedilol characterized by data selected from: X-ray powder diffraction pattern with peaks at about 4.3, 10.6, 11.1, 15.6, and 21.2±0.2 degrees two-theta; and DSC thermogram with endothermic peaks at about 60° C. and 113° C.

### BRIEF DESCRIPTION OF THE FIGURES

FIG. 1 illustrates a PXRD pattern for the carvedilol Form of the present invention.

FIG. 2 illustrates a DSC thermogram for the carvedilol Form of the present invention.

FIG. 3 illustrates a TGA thermogram for the carvedilol Form of the present -invention.

FIG. 4 illustrates a PXRD pattern for carvedilol Form II.

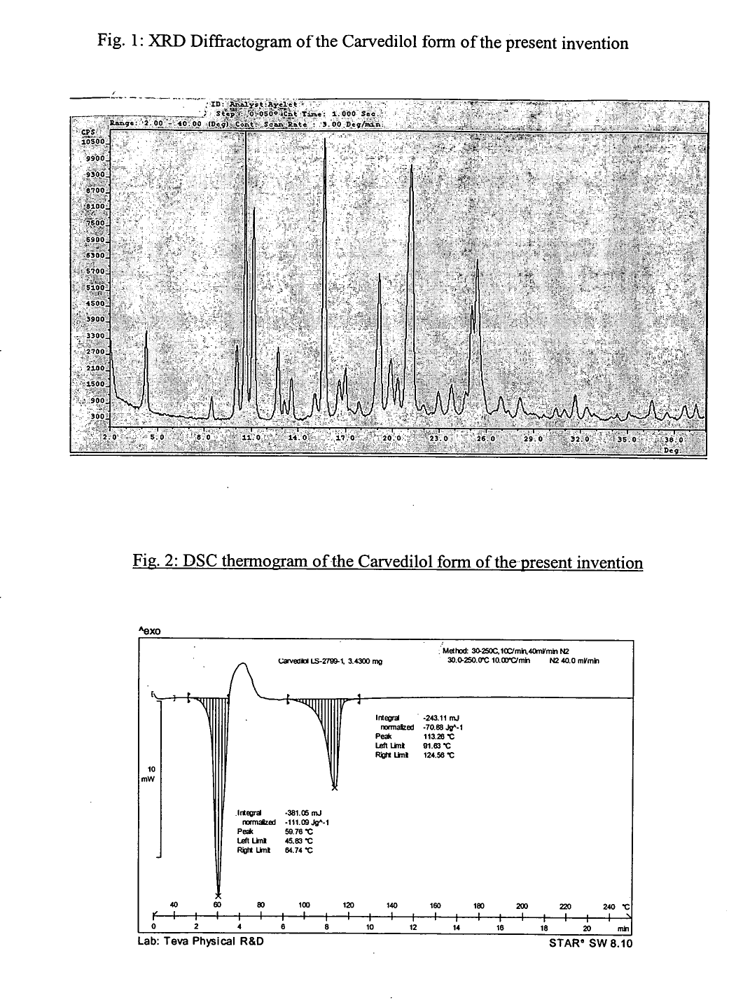

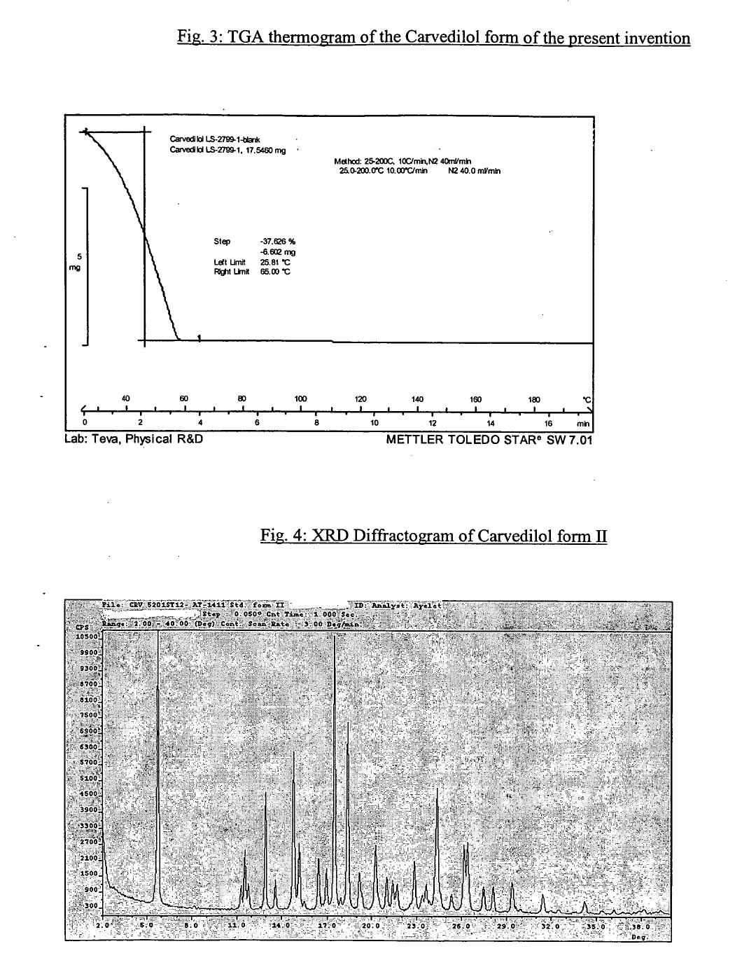

### DETAILED DESCRIPTION OF THE INVENTION

In one embodiment, the present invention provides a crystalline form of carvedilol characterized by data selected from: X-ray powder diffraction pattern with peaks at about 4.3, 10.6, 11.1, 15.6, and 21.2±0.2 degrees two-theta; and DSC thermogram with endothermic peaks at about 60° C. and 113° C.

The above crystalline form may be further characterized by X-ray powder diffraction pattern with peaks at about 8.5, 10.1, 12.7, 13.6, 16.6, 17.0, 19.1, 19.9, 20.3, 25.0, 25.4±0.2 degrees two-theta.

The crystalline form of the present invention may further be characterized by TGA showing a weight loss of about 40% at the temperature range of 25-60° C.

The crystalline form of the present invention may be an ethyl acetate solvate.

As used herein, the term “clean flask” refers to a flask, washed with an organic solvent. Preferably, the flask is washed with ethyl acetate to leave no traces of carvedilol seeds, which enables spontaneous crystallization of the crystals form of the present invention, when entering the supersaturation range.

In another aspect, the present invention provides a process for preparing the above crystalline form comprising: providing a solution of carvedilol in ethyl acetate, in a clean flask, cooling the solution to a temperature of less than about 1° C., to obtain a precipitate and recovering the crystalline form.

Preferably, the solution is provided at a temperature of about 70° C. to about 78° C., more preferably, at a temperature of about 70° C. to about 75° C.

Preferably, the solution is subjected to agitation.

Preferably, the solution is cooled to a temperature of about 0° C. to about 5° C.

In another aspect, the present invention provides a process for preparing a crystalline carvedilol characterized by X-ray powder diffraction pattern with peaks at about 5.9, 14.9, 17.6, 18.5 and 24.4±0.2 degrees two-theta (Form II) comprising drying crystalline carvedilol characterized by X-ray powder diffraction pattern with peaks at about 4.3, 10.6, 11.1, 15.6, and 21.2±0.2 degrees two-theta.

Preferably, the drying is performed at a temperature of about 30° C. to about 100° C., more preferably, at a temperature of about 40° C. to about 60° C., most preferably, at a temperature of about 50° C.

As one skilled in the art will appreciate, the time required to obtain carvedilol Form II will vary depending upon, among other factors, the amount of the wet carvedilol form of the present invention to be dried and the drying temperature, and can be determined by taking periodic XRDs.

The crystalline form of the present invention shows higher crystallinity compared with forms V and VI. The crystallinity is demonstrated by sharper XRD peaks and is an important factor that may influence the stability of the crystal form.

Pharmaceutical compositions of the present invention contain carvedilol form described above, optionally in mixture with other crystalline forms and/or other active ingredients such as hydrochlorothiazide. In addition to the active ingredient(s), the pharmaceutical compositions of the present invention can contain one or more excipients. Excipients are added to the composition for a variety of purposes.

Diluents increase the bulk of a solid pharmaceutical composition and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet can include excipients whose functions include helping to bind the active ingredient and other excipients together after compression. Binders for solid pharmaceutical compositions include at least one of acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, or starch.

The dissolution rate of a compacted solid pharmaceutical composition in the patient's stomach can be increased by the addition of a disintegrant to the composition. Disintegrants include, but are not limited to, alginic acid, carboxymethylcellulose calcium, carboxymethylcellulose sodium (e.g. Ac-Di-Sol@Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explota®) or starch.

Glidants can be added to improve the flow properties of non-compacted solid composition and improve the accuracy of dosing. Excipients that can function as glidants include colloidal silicon dioxide, magnesium trisilicate, powdered cellulose, starch, talc, and/or tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product form the dye. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and/or zinc stearate.

Flavoring agents and flavor enhancers make the dosage form more palatable to the patient. Common flavoring agents and flavor enhancers for pharmaceutical products that can be included in the composition of the present invention include, but are not limited to, maltol, vanillin, ethyl vanillin, menthol, citric acid, fumaric acid, ethyl maltol, or tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, carvedilol form described above and any other solid excipients are dissolved or suspended in a liquid carrier such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, or cetyl alcohol.

Liquid pharmaceutical compositions of the present invention can also contain a viscosity-enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch, tragacanth or xanthan gum.

Sweetening agents such as sorbitol, saccharin, sodium saccharin, sucrose, aspartame, fructose, mannitol and/or invert sugar can be added to improve the taste.

Preservatives and chelating agents such as alcohol, sodium benzoate, butylated hydroxy toluene, butylated hydroxyanisole and ethylenediamine tetraacetic acid can be added at levels safe for ingestion to improve storage stability.

A liquid composition according to the present invention can also contain a buffer such as gluconic acid, lactic acid, citric acid or acetic acid, sodium gluconate, sodium lactate, sodium citrate or sodium acetate.

Selection of excipients and the amounts to use can be readily determined by the formulation scientist based upon experience and consideration of standard procedures and reference works in the field.

The solid compositions of the present invention include powders, granulates, aggregates and compacted compositions.

Carvedilol form described above can be administered for treatment of congestive heart failure and hypertension (by any means that delivers the active ingredients) to the site of the body where beta-blocking activity exerts a therapeutic effect on the patient. For example, administration can be oral, buccal, parenteral (including subcutaneous, intramuscular, and intravenous) rectal, inhalant or ophthalmic. Although the most suitable route in any given case will depend on the nature and severity of the condition being treated, the most preferred route of the present invention is oral. Carvedilol Form VI, or the carvedilol crystalline form of the present invention, can be conveniently administered to a patient in oral unit dosage form and prepared by any of the methods well-known in the pharmaceutical arts. Dosage forms include solid dosage forms like tablets, powders, capsules, sachets, troches, or lozenges as well as liquid syrups, suspensions, or elixirs.

The active ingredient(s) and excipients can be formulated into compositions and dosage forms according to methods known in the art.

A composition for tableting or capsule filing can be prepared by wet granulation. In wet granulation some or all of the active ingredients and excipients in powder form are blended and then further mixed in the presence of a liquid, typically water that causes the powders to clump up into granules. The granulate is screened and/or milled, dried and then screened and/or milled to the desired particle size. The granulate can then be tabletted or other excipients can be added prior to tableting such as a glidant and or lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well suited to direct compression tableting include microcrystalline cellulose, spray dried lactose, dicalcium phosphate dihydrate and/or colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

Yet more particularly, a tablet can, for example, be formulated by blending and directly compressing the composition in a tablet machine.

A capsule can, for example, be prepared by filling half of a gelatin capsule with the above tablet composition and capping it with the other half of the gelatin capsule.

A simple parenteral solution for injection can, for example, be prepared by combining carvedilol Form VI, or the carvedilol crystalline form of the present invention, sterile propylene glycol, and sterile water and sealing the composition in a sterile vial under sterile conditions.

Capsules, tablets and lozenges and other unit dosage forms preferably contain a dosage level of about 1 mg to about 100 mg of carvedilol form described above.

The following examples are given for the purpose of illustrating the present invention and shall not be construed as limiting the scope or spirit of the invention.

### EXAMPLES

### Instruments

XRD

XRD diffraction was performed on X-Ray powder diffractometer, Scintag model X'TRA, Cu-tube, solid state detector. Scanning parameters include: Range: 2-40 deg. 2θ, Continuous scan, Rate: 3.00 deg./min.

Thermal Analysis

DSC thermogram was performed on DSC821e, Mettler Toledo, sample weight: 3 mg, and heating rate: 10° C./min. The crucible was crimped and punched.

TGA thermogram was performed on Mettler TG50, sample weight: 18 mg, and heating rate: 10° C./min.

### Example 1

### Process for the Preparation of Carvedilol Form of the Present Invention

50 g of Carvedilol and 500 ml of Ethyl Acetate are put into clean flask, the slurry is heated to temperature higher than 70° C. to get full dissolution. The solution is cooled to about 0-5° C. At temperature of about 5-10° C. spontaneous precipitation occurred. The solid substance is filtered and washed by ethyl acetate. The wet substance obtained is the crystal form of the present invention.

### Example 2

### Conversion of the Crystal Form of the Present Invention to Form II

The Carvedilol crystal form of the present invention was prepared according to example 1. The wet substance was dried at about 50° C. XRD shows form II content for the dry substance.

1. A crystalline form of carvedilol characterized by data selected from:
X-ray powder diffraction pattern with peaks at about 4.3, 10.6, 11.1, 15.6 and 21.2±0.2 degrees two-theta; and
DSC thermogram with endothermic peaks at about 60° C. and 113° C.

2. The crystalline form of claim 1, characterized by X-ray powder diffraction pattern with peaks at about 4.3, 10.6, 11.1, 15.6 and 21.2±0.2 degrees two-theta.

3. The crystalline form of claim 2, further characterized by X-ray powder diffraction pattern with peaks at about 8.5, 10.1, 12.7, 13.6, 16.6, 17.0, 19.1, 19.9, 20.3, 25.0, 25.4±0.2 degrees two-theta.

4. The crystalline form of claim 1, further characterized by DSC thermogram with endothermic peaks at about 60° C. and 113° C.

5. The crystalline form of claim 1, further characterized by TGA showing a weight loss of about 40% at the temperature range of 25-60° C.
